(19) United States
(12) Patent Application Publication
(10) Pub. No.: US 2008/0031916 A1
(43) Pub. Date: Feb. 7, 2008

(54) Dendrimer cross-linked collagen

(76) Inventors: Heather Sheardown, Nobleton (CA); Xiaodong Duan, Hamilton (CA); Marta Princz, Hamilton (CA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/789,558

(22) Filed: Apr. 24, 2007

Related U.S. Application Data
(60) Provisional application No. 60/794,116, filed on Apr. 24, 2006.

(51) Int. Cl. A61K 9/10; A61K 31/715; A61P 19/04; C07K 14/78; A61K 38/00

(52) U.S. Cl. 424/423; 424/484; 514/12; 514/2; 514/54; 514/8; 530/356

(57) ABSTRACT

Dendrimer-crosslinked collagen is provided which is particularly suitable for use as a tissue engineering scaffold. The dendrimer-crosslinked collagen can also incorporate biomolecules to enhance its utility as a tissue engineering scaffold.

### FIELD OF THE INVENTION

The present invention relates to a novel method of crosslinking collagen using dendrimers, to the resulting dendrimer cross-linked collagen matrix and to modification of the dendrimer collagen matrix to incorporate biomolecules.

### BACKGROUND OF THE INVENTION

Collagen, the most abundant protein in the body, is the major constituent of connective tissues. As such, it has been widely applied in biomaterials applications as, for example, a wound dressing, a matrix for controlled release of active agents or a tissue-engineering scaffold [1-5]. Collagen as a biomaterial offers such advantages as biocompatibility, low toxicity to most tissues, and well documented structural, physical, chemical and immunological properties. It can be readily isolated and purified in large quantities and can be processed into a variety of forms [6]. Collagen scaffolds have been applied to the engineering of such tissues as cartilage [7], cornea [8-10] and dermal skin [11].

However, in its purified form, collagen forms a weakly crosslinked thermo gel. Therefore, for tissue engineering applications, covalent intermolecular crosslinks between collagen molecules in macromolecular fibrils using appropriate biocompatible molecules is essential for the development of stable materials with a high degree of mechanical integrity. While glutaraldehyde has been widely used as a collagen crosslinking agent [12,13] and is generally thought to result in one of the highest crosslink densities [14], cytotoxicity, and a lack of understanding of the mechanisms of the reaction make it desirable to find alternative effective crosslinking mechanisms [15,16].

Alternative procedures have been explored for physically crosslinking collagen, including dehydrothermal treatment, ultraviolet irradiation [17,18] as well as novel chemical crosslinkers including diisocyanates, acyl azide [19], and 1-ethyl-3-(3-dimethyl aminopropyl) carbodiimide (EDC) [20]. Most of these crosslinkers including glutaraldehyde, hexamethylene diisocyanate and acyl azide are “bridge-forming” meaning that the crosslinker acts as a chemical “bridge” between collagen molecules. However, with EDC, “zero-length” crosslinks are formed, meaning that the collagen molecules are linked directly. Since amines are the limiting functional groups in collagen for crosslinking [20], the use of amine rich compounds in combination with EDC has been examined. Collagen gels crosslinked in the presence of diamines [21] showed little improvement in mechanical properties and biological stability relative to EDC-crosslinked controls. This is possibly due to the relatively short length of the diamines selected and potentially a lack of adequate amounts of free amine groups in the diamines to sufficiently enhance the reaction. In comparison, longer and more amine rich lysine containing peptides have shown promising results as agents to facilitate collagen crosslinking [22]. Others have used multifunctional amines for crosslinking of polymers other than collagen [23].

The extracellular matrix (ECM) is the natural scaffold for the cells, acting as a mechanical support and creating a microenvironment to which the cells can respond. Constructing a matrix or scaffold which simulates the ECM environment is therefore desirable and a widely used strategy in tissue engineering. Such a scaffold has the potential to promote cell growth and to restore key functions to damaged tissues and organs. To mimic the high proportion of collagen present in most native tissues, collagen scaffolds are widely used in tissue engineering.

However, the biological function of these tissues is in large part due to the presence of other extracellular components. For example, the extracellular matrix protein, laminin, has been previously used to promote neurite growth [24]. The YIGSR sequence of laminin has been incorporated into tissue engineering scaffold materials to promote peripheral [25], and central [26] nerve regeneration. In corneal applications, YIGSR grafted to a collagen-acrylate copolymer scaffold has been shown to promote human corneal epithelial stratification and neurite ingrowth [27].

The dynamic interactions of collagen scaffolds with the surrounding biological environment in vivo make it desirable to incorporate additional biological functionality into a crosslinked collagen matrix in the form of cell adhesion molecules like peptides and growth factors. However, most currently available crosslinking technologies, such as those described above, will not permit functionalization of the matrix without potentially altering the biological properties of the collagen itself. Thus, it would be desirable to develop methodology which results in mechanically strong collagen matrices and permits the incorporation of biological functionality into a collagen matrix without altering the properties of the matrix.

### SUMMARY OF THE INVENTION

A novel collagen matrix has now been developed in which collagen solutions are cross-linked with multifunctional dendrimers resulting in mechanically strong collagen hydrogels with high crosslinking densities. The dendrimer crosslinked collagen showed unique thermal characteristics, with high temperature transitions and multiple denaturation temperature peaks in contrast to other crosslinked collagens. The dendrimer collagen matrix is particularly suitable for use as a tissue engineering scaffold in vitro and in vivo and for the incorporation and delivery of biomolecules in vivo.

Thus, in one aspect of the present invention, a dendrimer crosslinked collagen matrix is provided.

In another aspect, a method of preparing dendrimer crosslinked collagen is provided comprising the steps of incubating a collagen solution with a dendrimer solution in the presence of an agent capable of facilitating the crosslinking for a period of time suitable to achieve the desired amount of crosslinking.

In another aspect of the present invention, dendrimer crosslinked collagen is provided for use as a tissue engineering scaffold.

These and other aspects of the invention will become apparent from the following detailed description and figures in which:

### BRIEF DESCRIPTION OF THE FIGURES

FIG. 1 graphically illustrates the relative equilibrium water uptake of various collagen samples, including uncrosslinked, EDC crosslinked, glutaraldehyde crosslinked and dendrimer crosslinked collagen;

FIG. 2 graphically illustrates the denaturation temperatures (T_{d}) of collagen samples measured by DSC before and after crosslinking;

FIG. 3 graphically illustrates the relative degradation percentage of collagen samples during exposure to a collagenase solution (pH 7.4, 37° C., 24 h);

FIG. 4 graphically illustrates the number of activated carboxylic acid groups in various collagen samples;

FIG. 5 is a schematic of a generation 2 polypropyleneimine dendrimer;

FIG. 6 graphically illustrates visible light transmission through the various collagen samples as measured spectrophotometrically;

FIG. 7 illustrates TEM photos of various collagen samples (Magnification 20,000, bar=200 nm) including uncrosslinked (A), EDC-crosslinked (B), glutaraldehyde-crosslinked (C) and dendrimer crosslinked (D) collagen samples.

FIG. 8 graphically compares Young's modulus of various crosslinked collagen samples (a); the maximum load measured for various crosslinked collagen samples (b); and the displacement at maximum load of different collagen samples (c);

FIG. 9 graphically compares the effect of collagen concentration on the mechanical properties of various collagen samples;

FIG. 10 graphically illustrates the mechanical properties of dendrimer crosslinked collagen gel samples;

FIG. 11 (a-d) are representative photomicrographs of human corneal epithelial cells grown on various collagen samples at 120 minutes;

FIG. 12 (a-d) are representative photomicrographs of human corneal epithelial cells on the collagen gels after 4 days of culture;

FIG. 13 graphically illustrates the cell quantification analysis results of various collagen samples at a) 120 minutes and b) 3 and 4 days;

FIG. 14 is an H-NMR spectra of YIGSR (a), dendrimer G₂ (b), and YIGSR-modified dendrimer (c);

FIG. 15 is a MALDI-TOF spectra of a) a dendrimer and b) a YIGSR-modified dendrimer;

FIG. 16 is a comparison of mechanical properties (a, Young's modulus; b, Maximum load) of YIGSR-modified (6.4 μg/mg collagen) and unmodified collagen samples;

FIG. 17 illustrates HCEC adhesion on YIGSR-modified collagen gels after 2 hours of culture;

FIG. 18 illustrates HCEC proliferation on YIGSR-modified collagen gels after 2 days of culture;

FIG. 19 illustrates HCEC proliferation on YIGSR-modified collagen gels after 4 days of culture;

FIG. 20 illustrates HCEC proliferation on YIGSR-modified collagen gels determined by Cyquant assay;

FIG. 21 graphically illustrates DRG neurite extension on YIGSR-modified collagen gels compared with an unmodified control; and

FIG. 22 illustrates DRG nerve cell in-growth on an unmodified control (left) and YIGSR-modified (right, 6.4 μg/mg collagen) collagen gels. Neurites extended longer on YIGSR-modified collagens.

### DETAILED DESCRIPTION OF THE INVENTION

Dendrimer crosslinked collagen is herein provided. Dendrimer functional groups permit collagen crosslinking to occur and result in a mechanically strong collagen matrix. The dendrimer functional groups are also useful to bind biomolecules and thereby result in incorporation of biomolecules into a dendrimer collagen matrix without significantly altering the crosslinking density or the biological properties of the dendrimer collagen matrices. Thus, dendrimer crosslinked collagen additionally provides a means of delivering biomolecules to a desired site in vivo.

The term “dendrimer” is used herein to refer to a polymeric molecule composed of a repeating monomer (or dendrimer core). A dendrimer has a branching shape and end groups that are functional for cross-linking collagen. Depending on the dendrimer core (central or core monomer), the dendrimer may have 3, 4, 6, 8 or more branches and therefore is multifunctional. There are a large number of molecules which can be used as the core monomer for a dendrimer. Examples of suitable dendrimer cores for use in the present invention include an alkyl-diamine such as ethyl-diamine and propyl-diamine, to form substituted diamines; an alkyl dicarboxylic acid such as malonic acid, succinic acid and adipic acid; and aldehyde-terminated dendrimers such as PAMAM. As used herein the term “alkyl” is not limited with respect to carbon number, as one of skill in the art will appreciate, and may include a C₁-C₅ alkyl group, for example, an alkyl group having 1-5 carbon atoms.

The term “biomolecule” is used herein to refer to an entity that is biologically active or functional to provide a required linkage, stimulation or therapy, and thus, may be selected from a wide range of molecules, as one of skill in the art will appreciate. Thus, a biomolecule for use in the present application may comprise, but is not limited to, a peptide such as cell adhesion peptide YIGSR, RGD, IKVAV and RNIAEIIKDI; a glycoprotein such as laminin; a protein such as a growth factor; a polysaccharide such as heparin; and a naturally occurring or synthetic linker, therapeutic, growth stimulant or cell adhesion stimulant.

The dendrimer crosslinked collagen of the present invention is prepared by incubating a suitable dendrimer with a collagen solution under conditions which result in polymerization. In order for the crosslinking to occur, a facilitating agent must be added to the reaction mixture. The facilitating agent is any agent capable of causing the crosslinking between collagen and the dendrimer to occur. For example, where an amine-terminated dendrimer is used, crosslinking is facilitated by a carbodiimide, such as EDC or DDC (N,N′-Dicyclohexylcarbodiimide) and optimal conditions for this polymerization include, but are not necessarily restricted to, a pH of between 5.0 and 6.0, preferably a pH of 5.5, and incubation overnight at 37° C. Where a carboxyl-terminated dendrimer is used, a carbodiimide or other facilitating agent may be used under polymerization conditions as described above. In any case, it is desirable to degas the collagen to maintain the mechanical properties of the resulting collagen matrix. A stability agent, such as N-hydroxysulfosuccinimide (NHS) or 1-Hydroxybenzotriazole (HOBT), may also be used in the crosslinking reaction. Stability agents include hydrophilic active groups that react rapidly with amines on target molecules and increase the stability of the active intermediate which ultimately reacts with the attacking amine. Although not necessary for crosslinking to occur, stability agents, such as NHS, significantly increase the yield of crosslinked product.

The amount of dendrimer and collagen used to make the crosslinked product is not particularly restricted, and will depend on the nature of the dendrimer used for cross-linking (the greater the number of amine or carboxyl cross-linking groups on the dendrimer i.e, the generation of the dendrimer, the less the amount of dendrimer required). It will also depend on the desired cross-linked product. If a crosslinked product with free/available cross-linking groups is desired, then an amount of collagen and dendrimer is used in which dendrimer functional groups are in excess to cross-linking groups of the collagen. The greater the amount of collagen used in relation to dendrimer, the greater the number of dendrimer amine groups that are utilized in the crosslinking reaction. Generally a ratio of collagen to dendrimer of 10:1 or less (e.g. 5:1) results in a significant excess of dendrimer functional groups and further increases of dendrimer, thus, would not increase the level of crosslinking.

The use of multifunctional dendrimers, i.e. multi-branch dendrimers for making a crosslinked collagen matrix advantageously provides an increased number of functional groups, e.g. free amine groups, available for crosslinking with activated carboxylic acid groups of the collagen relative to carbodiimide and glutaraldehyde crosslinking counterparts. While not wishing to be restricted to any particular mode of action, it is believed that the dendrimers act as “bridges” linking the collagen molecules. Furthermore, in addition to introducing a large number of amine groups, dendrimers provide groups that are more accessible for crosslinking than those in the collagen. Therefore, the use of dendrimers for collagen crosslinking increases both the extent of crosslinking with collagen, and quality of the crosslinking (bridge linkage versus “zero-length” crosslinking).

The dendrimer crosslinked collagen product has features of high mechanical strength and high crosslinking densities in comparison to crosslinked collagen counterparts. High crosslinking densities are evidenced by its unique thermal characteristics of high temperature transitions and multiple denaturation temperatures which are not evident in other crosslinked collagens. High mechanical strength is evidenced by Young's modulus of at least about 0.2 Mpa, and preferably, at least about 1.0 Mpa, as well as displacement at maximum load of less than about 3.0 mm, and preferably, less than 2.0 mm.

The dendrimer collagen matrix may additionally incorporate a biomolecule to enhance the utility of the matrix. The biomolecule may be incorporated into the dendrimer collagen matrix by linkage to the dendrimer prior to crosslinking of the dendrimer to the collagen. The linkage of the biomolecule to the dendrimer may vary with the nature of the biomolecule; however, generally, this linkage involves incubation of the biomolecule with the dendrimer under conditions suitable to catalyze linkage of the biomolecule to the functional groups on the dendrimer also utilized for collagen cross-linking. If the biomolecule contains the same functional groups as the collagen crosslinking groups, it may be added into the reaction mixture during the collagen crosslinking reaction. The biomolecule may require modification to incorporate a linker that will allow ready linkage of the biomolecule to the dendrimer functional group. Examples of suitable linkers are known to those of skill in the art and include, for example, carboxylic acids, amines, hydroxyls or hydroxyl amines. In addition, additives which facilitate the linkage of the biomolecule to the dendrimer may be used, such as EDC to facilitate amine-carboxylic acid linkages, as one of skill in the art will appreciate. The amount of biomolecule admixed with dendrimer is such that the functional groups on the dendrimer are in excess of the biomolecule so that functional groups remain available on the dendrimer for subsequent or simultaneous collagen cross-linking.

Alternatively, the biomolecule may be linked to the dendrimer collagen matrix following the crosslinking reaction. This linkage is conducted by incubating the matrix with the biomolecule under conditions of temperature and pH which facilitate the bonding of the biomolecule to the matrix, and particularly, to available functional groups of the dendrimer. The incubation may be conducted in the presence of components which facilitate the biomolecule-dendrimer linkage to occur as previously described. The amount of biomolecule in this case in not restricted as the dendrimer collagen matrix is formed and the biomolecule links with available dendrimer functional groups.

The dendrimer collagen matrix, optionally incorporating a selected biomolecule, is useful as a tissue engineering scaffold in vitro and for targeted sites in vivo, to encourage the growth of tissue for replacement or repair of damaged tissue, as well as for the delivery of therapeutic agents to a diseased region. The incorporation of biomolecules into the present collagen matrix further enhances their use as tissue engineering scaffolds.

In one embodiment, polypropyleneimine octaamine dendrimers can be used to generate dendrimer crosslinked collagen with mechanical properties appropriate for its use as a tissue engineering scaffold. The dendrimer crosslinked collagen of the present invention may be used as a scaffold in vitro, to grow tissue for subsequent implant or transplantation. Alternatively, the dendrimer crosslinked collagen can be inserted at a desired site in vivo to promote or regenerate tissue growth.

In another embodiment, dendrimer crosslinked collagen incorporating a biomolecule can be used as a tissue engineering scaffold in connection with various cells/tissues. The biomolecule, for example, a cell adhesion factor, can advantageously promote or accelerate cell adhesion and growth. As exemplified in the specific examples herein, dendrimer crosslinked collagen incorporating a cell adhesion biomolecule promoted cell adhesion and proliferation of corneal epitheal cells and neurite cells in comparison to other crosslinked collagen samples.

Embodiments of the invention are described by reference to the following specific examples which are not to be construed as limiting.

### EXAMPLE 1

### Preparation of Dendrimer Cross-Linked Collagen and Properties Thereof

Collagen Crosslinking

Uncrosslinked collagen controls were prepared by neutralizing a 0.4% type I collagen solution from rat tail tendon (Becton Dickinson, Mississauga ON) with 0.1N NaOH and subsequent incubation in a 37° C. oven overnight to gel. For preparation of the EDC-crosslinked collagen, 5 ml of a 0.4% type I collagen solution was added to a pre-cooled glass vial with 1 ml 1-ethyl-3-(3-dimethyl aminopropyl) carbodiimide (EDC)/N-hydroxysuccinimide (NHS) in aqueous solution (2.3 mg EDC (Sigma Aldrich, Oakville ON), 1.4 mg NHS (Sigma Aldrich, Oakville ON)) and mixed thoroughly. The pH was adjusted to 5.5 with 0.1 N NaOH and/or 0.1 N HCl and the resultant solution was placed in a 37° C. oven overnight to gel. This pH has been suggested as the optimum pH for activation of carboxylic acid groups with EDC for subsequent functionalization [15].

Glutaraldehyde crosslinked collagen was prepared as a control. After neutralization with 0.1N NaOH, the collagen solution was mixed with an aqueous solution of glutaraldehyde (Sigma Aldrich, Oakville ON) to a final concentration of 0.9%. The solution was left in a 37° C. oven overnight to gel and crosslink.

Diamine (ethylene diamine), triamine (tris (2-aminoethyl amine) and dendrimer crosslinked collagens were prepared using the following procedure. To 5 mL of a 0.4% type I collagen solution was added 1 mL of an aqueous solution containing 2.3 mg EDC, and the multifunctional dendrimer crosslinking agent (Sigma Aldrich, Oakville ON) in an amount dependant on the weight ratio of collagen to dendrimer, and 1.4 mg NHS. The pH of the solution was adjusted to 5.5 and the solution was placed in a 37° C. oven overnight for crosslinking and gelation. Three generations of polypropyleneimine dendrimers, generation 1 (G₁) with 4 amine terminal arms, generation 2 (G₂) with 8 arms and generation 3 (G₃), with 16 arms, were used in this study. Different ratios of collagen to dendrimer were studied to examine the effect of dendrimer amount on crosslinking in the resultant gels.

Since the gels prepared from the dilute collagen solutions (0.4% w/v) were weak and difficult to handle and characterize, all of the gels were freeze-dried to obtain sponges for further characterization. The sponges were stored at 4° C. until characterization.

Characterization of Collagen Samples

Water Uptake

Collagen sponge samples (n>3, approximately 5 mg) were dried completely overnight, weighed and incubated in 3 mL of PBS (pH 7.2) at room temperature for 1 hour. It was determined that 1 hour was sufficient for these highly porous gels to reach equilibrium. The wet weight was then determined and the absolute water uptake calculated using the equation:

$$Water Uptake ( \% ) = \frac{{W}_{w} - {W}_{d}}{{W}_{d}} \times 100 \%$$

where W_{w} and W_{d} are the wet and dry weights as measured respectively. The results were normalized to the uncrosslinked collagen control, at 100%.
Differential Scanning Calorimetry (DSC)

The denaturation temperatures of the collagen samples were determined using a TA DSC instrument. Denaturation temperature has been previously suggested to provide information about the crosslinking density of collagen samples [14,28]. Collagen samples (2 mg) were immersed in 30 μl of demineralized water in aluminum hermetic pans for 2 hours at room temperature. Hermetic pans containing 30 μl demineralized water only were used as the reference. A heating rate of 5° C./min was applied in a temperature range from 15 to 100° C. and the endothermic peak(s) of the thermogram was monitored and recorded. While heating rate can affect the observed denaturation values, similar temperatures were obtained with a heating rate of 2° C./minute.

Collagenase Assay

A collagenase assay [12,22] was performed to further examine the crosslinking in the samples and provide information about their biological stability. Collagen samples with a dry weight of approximately 5 mg were incubated for 1 hour in 0.1M Tris-HCl (pH7.4) containing 0.05M CaCl₂ at 37° C. Subsequently, 200 U of bacterial collagenase (Clostridium histolyticum, EC 3.4.24.3, Sigma Chemical Co.) in 1 mL of 0.1M Tris-HCl (pH7.4) was added. After 24 h at 37° C., the reaction was stopped by the addition of 0.25M EDTA and cooling the mixture on ice. The mixtures were then centrifuged and the supernatant analyzed for hydroxyproline (Hyp) [13]. Briefly, aliquots of standard Hyp (2-20 μg) prepared from a stock solution and 10 μl supernatant were mixed gently with sodium hydroxide (2N). The samples were hydrolyzed by autoclaving at 120° C. for 20 min. Chloramine-T was subsequently added to the above solution, mixed gently, and the oxidation was allowed to proceed for 25 minutes at room temperature. This was followed by the addition of Ehrlich's aldehyde reagent (p-dimethylaminobenzaldehyde dissolved in n-propanol/perchloric acid 2:1 v/v) to each sample and the development of the chromophore by incubating the samples at 65° C. for 20 min. The absorbance of each sample was read at 550 nm using a spectrophotometer and compared to a standard calibration curve to quantify the amount of Hyp.

Measurement of Activated Carboxylic Acid Groups

As previously described [15], the total number of NHS-activated carboxylic acid groups prior to crosslinking and the amount available after the crosslinking reaction were determined. Briefly, the free amine groups of collagen samples were blocked using the acylating agent, acetic acid NHS ester (HAc-NHS). An aqueous solution containing HAc-NHS was added to a 0.4% collagen solution (NHS:NH₂=5:1) and the reaction allowed to proceed for 5 hours at room temperature (pH˜6.5 to 7.5). The collagen samples with blocked amine groups were reacted with EDC and NHS at pH 5.5 as described. The samples, including those with blocked amine groups, were then washed for 1 hour in 20 mL of 0.2 M NaH₂PO₄ buffer (pH 4.5) to remove unreacted NHS, and subsequently immersed in 1 mL of 0.1 M Na₂HPO₄ buffer (pH9.1) for a period of 2 hours. The amount of NHS released was measured spectrophotometrically at 260 nm assuming ε=9700M⁻¹cm⁻¹.

Results

Crosslinking of Low Concentration Collagen Gels

The general appearance of the various collagen gels prepared is summarized in Table 1 below.

TABLE 1
Macroscopic Appearance and Relative Mechanical Properties of Gels

| | | | Relative | |
|---|---|---|---|---|
| | | | Mechanical | Gel |
| Collagen Sample | Crosslinker | pH | Strength | Appearance |
| Uncrosslinked | N/A | 7.5 | Fair | Translucent |
| EDC | EDC + NHS | 5.5 | Poor | Transparent |
| Diamine | EDC + NHS + ED | 5.5 | Poor | Transparent |
| Triamine | EDC + NHS + TA | 5.5 | Poor | Transparent |
| Glutaraldehyde | Glutaraldehyde | 7.5 | Poor | Translucent |
| G₁ Dendrimer | EDC + NHS + | 5.5 | Poor | Transparent |
| | dendrimer | | | |
| G₂, G₃ Dendrimer | EDC + NHS + | 5.5 | Good | Transparent |
| | dendrimer | | | |

Results were generally quite consistent if bubble formation in the gels was minimized. The EDC, diamine and triamine crosslinked collagen gels had relatively poor mechanical properties compared to the other gels. The glutaraldehyde crosslinked gels were also relatively mechanically weak. In comparison, the G₂ and G₃ dendrimer crosslinked gels exhibited comparatively good mechanical strength. The collagen solution without the addition of the EDC and with the addition of dendrimers, did not gel at pH 5.5, the optimal condition for the carbodiimide crosslinking reaction. This result provides evidence for the carbodiimide crosslinking and for the necessity of the carbodiimide in the crosslinking of collagens with dendrimers. The gels were freeze dried for further characterization.

Water Uptake

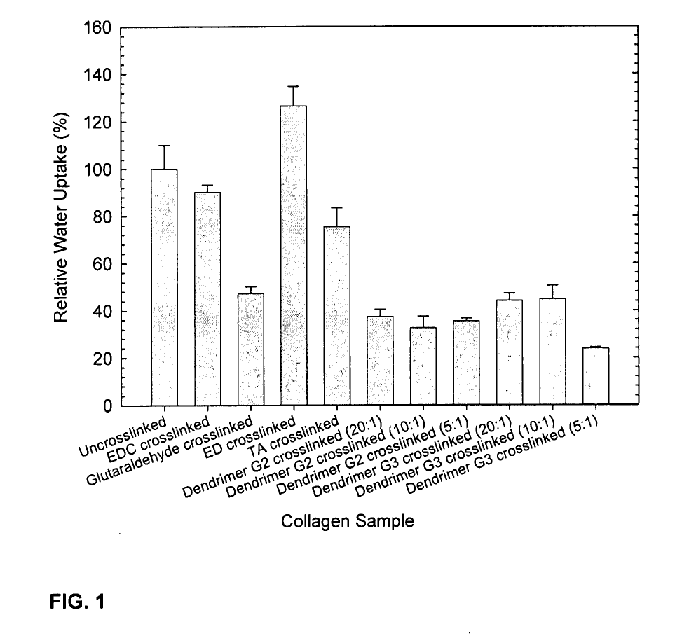

Water uptake results for the various collagen samples are illustrated in FIG. 1. The water uptake of EDC-crosslinked collagen decreased very little relative to the thermo-gelled sample (approximately 10%), possibly due to the “zero-length” crosslinking nature and suggesting that EDC crosslinking alone may not be suitable for generating highly crosslinked samples. Similarly, the combination of a di- or tri-amine and EDC had little impact on the water uptake relative to the control samples. However, as expected, the glutaraldehyde-crosslinked collagen samples had a much lower water uptake, with a decrease of approximately 50% relative to the thermo-gelled controls, indicating a higher degree of crosslinking in these samples. Similarly, the G₂ and G₃-dendrimer crosslinked collagen samples showed similar decreases in the water uptake of between 50% and 70%, inferring that significant crosslinking was occurring with the use of a combination of EDC and dendrimers. The G₁ dendrimer crosslinked samples showed similar results to the di- and tri-amine-crosslinked samples.

Water uptake results suggest that altering the ratio of G₂ and G₃ dendrimers to collagen had a small but relatively insignificant effect on the crosslinking. Furthermore, somewhat surprisingly based on the results with the G₁ dendrimers, the use of a G₃ dendrimer with 16 functional amine groups did not decrease water uptake relative to the use of G₂ dendrimers with only 8 functional groups. It is likely that the additional functional groups present on the G₃ dendrimers cannot effectively participate in crosslinking due to steric factors.

Differential Scanning Calorimetry (DSC)

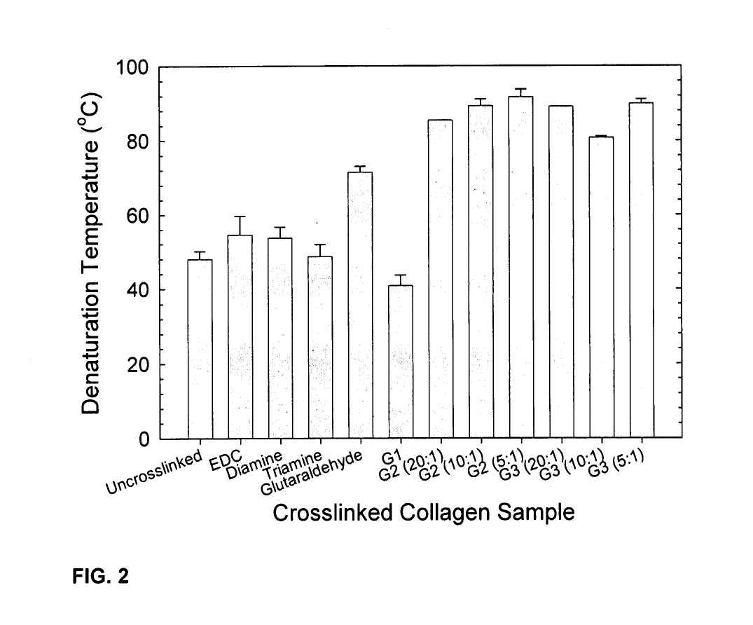

Measurement of denaturation (shrinkage) temperatures (T_{d}) of collagen samples by DSC is commonly used to evaluate the efficiency and extent of crosslinking [14,28]. In general, crosslinking of the collagen gels with various crosslinkers resulted in an increase in the denaturation temperature as shown in FIG. 2. The EDC-crosslinked collagen showed only a slight increase in the denaturation temperature from 48° C. in the uncrosslinked sample to 55° C. Denaturation temperatures of 54° C., 48° C. and 45° C. were noted for the ethylene diamine, triamine and G₁ dendrimer crosslinked samples, respectively. Consistent with the water uptake and macroscopic uptake results, these results suggest that the level of crosslinking in these samples was not significant. Glutaraldehyde-crosslinking, which involves reaction with the free amine groups present in collagen, resulted in a further increase of the T_{d} to 71° C. Higher T_{d} values of between 80° C. and 90° C. were noted following crosslinking with the G₂ and G₃ dendrimers at pH 5.5. This indicates that the use of dendrimers with higher numbers of functional amine groups for crosslinking may result in a higher crosslinking density than that obtained using glutaraldehyde. While there was a trend toward higher denaturation temperatures with increased amounts of dendrimer with the G₂ dendrimer-crosslinked samples, differences were not significant. A similar trend was not observed with the G₃ dendrimer-crosslinked samples. Furthermore, there were no clear differences in the observed denaturation temperatures between the G₂ and G₃ dendrimer-crosslinked samples.

A unique characteristic feature of G₂ and G₃ dendrimer-crosslinked collagens but not the G₁ dendrimer sample was noted during DSC peak assignment. These samples showed multiple peaks in the DSC scans as noted in Table 2 below.

TABLE 2
Presence of multiple denaturation peaks in dendrimer
crosslinked collagen

| | Denaturation Temperature | | | | |
|---|---|---|---|---|---|
| Sample | (° C.) | | | | |
| Uncrosslinked | 47.9 | | | | |
| EDC crosslinked | 54.7 | | | | |
| Glutaraldehyde crosslinked | 71.5 | | | | |
| G2 crosslinked (20:1) | 51.4 | 85.4 | | | |
| G2 crosslinked (10:1) | 40.0 | 82.2 | 89.3 | | |
| G2 crosslinked (5:1) | 37.8 | 51.0 | 67.4 | 80.4 | 92.0 |
| G3 crosslinked (20:1) | 51.5 | 67.8 | 89.1 | | |
| G3 crosslinked (10:1) | 41.1 | 70.3 | 80.8 | | |
| G3 crosslinked (5:1) | 45.4 | 68.1 | 80.6 | 89.9 | |

The presence of these multiple peaks could be the result of complexity and heterogeneity in the dendrimer-crosslinked samples due to the multifunctionality of the dendrimers. These peaks were denaturation peaks as confirmed by a second DSC; since the denaturation of collagen is an irreversible process, the denaturation peak(s) present in the first DSC scan did not appear in subsequent scans.

Collagenase Assay

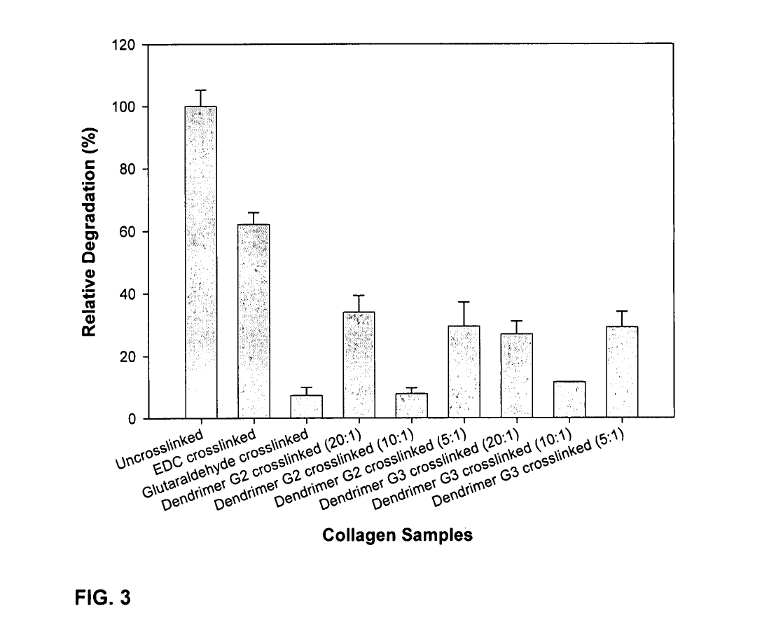

The degradation and therefore biological stability of the collagen samples was studied by exposing materials to a collagenase solution. The degraded collagens were quantified by analysis of hydroxyproline release, a major component of collagen. The results are shown in FIG. 3 as percentages of degraded collagen relative to uncrosslinked samples. Following crosslinking, the degradation of the samples decreased to various extents depending on the nature of the crosslinkers used. Approximately 60% of the EDC-crosslinked collagen was degraded, possibly due to the less efficient “zero-length” crosslinking that occurs with this method. Consistent with the results of others, glutaraldehyde showed significant ability to improve the biostability of the collagen samples. As little as 7% of the glutaraldehyde-crosslinked collagens were degraded under identical conditions. With the aid of G₂ and G₃ dendrimers, the carbodiimide-crosslinking could achieve comparative biostability to that noted with glutaraldehyde. The results suggest that collagen to dendrimer in a 10:1 ratio (w/w) resulted in the greatest improvement in the proteolytic resistance of the collagen in these samples. However, the stability of all of the dendrimer-crosslinked samples was similar.

Measurement of Activated Carboxylic Acid Groups

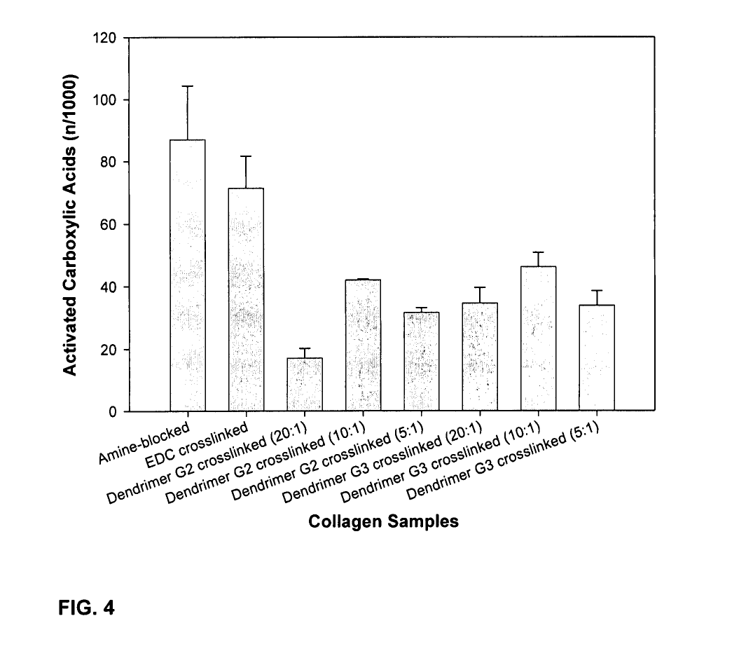

To provide further evidence of reaction and to determine the extent of this reaction, the number of activated carboxylic acid groups in carbodiimide-crosslinking reactions was monitored. The results are shown in FIG. 4. The difference between the number of crosslinked samples and that of the amine-blocked collagens was the amount of activated carboxylic acid groups consumed during the crosslinking reactions. Analysis of an amine blocked collagen sample suggests that the total number of activated carboxylic acid groups available for crosslinking was 87 per 1000. This is slightly lower than the estimate for total number of carboxylic acid groups of 120 per 1000, suggesting that the EDC does not activate 100% of the available carboxylic acid groups in the collagen or that not all of the amide groups were hydrolyzed. The EDC crosslinked collagen had 71/1000 activated carboxylic acid groups suggesting that 16/1000 had been consumed by the crosslinking reaction. In the G₂ and G₃ dendrimer crosslinked samples, the number of the consumed carboxylic groups was between 40 and 69 per 1000, clearly demonstrating that the introduction of dendrimers into the EDC crosslinking reaction resulted in crosslinking via the carboxylic acid groups and improved the extent of the reaction, likely due to large amount of free amine groups of dendrimers. Again, no clear trend was observed with changes in the weight ratios of collagen to dendrimers and G₂ versus G₃.

### EXAMPLE 2

### Utility and Biocompatability of Dendrimer Crosslinked Collagen

Collagen Gel Preparation

All the reagents used were purchased from Sigma Aldrich (Oakville ON) except when otherwise specified. Concentrated collagen suspensions, the generous gift of Inamed Corporation (USA), consisted of pepsin-digested bovine cornium purified predominantly type I collagen with less than 20% type III collagen. The 6% suspension was in phosphate buffered saline, pH 7.0-7.6. The suspensions were acidified with 1N HCl and diluted to make clear collagen solutions prior to further treatment.

A thermally crosslinked collagen control was prepared by neutralizing the collagen solution with 1N NaOH and subsequent incubation in a 37° C. oven overnight in order to allow for gelation to occur. EDC-crosslinked collagen gels were prepared by mixing the collagen solution with 1-ethyl-3-(3-dimethyl aminopropyl) carbodiimide (EDC)/N-hydroxysuccinimide (NHS) aqueous solution (molar ratio of EDC:NHS:COOH=5:5:1) in pre-cooled syringes on ice. The pH was subsequently adjusted to 5.5 with 1 N NaOH and/or 1 N HCl and the resultant solution was injected into glass moulds in a 37° C. oven overnight to gel.

Glutaraldehyde-crosslinked collagen gels were prepared as positive controls. After neutralization with 1N NaOH, the collagen solution was mixed with an aqueous solution of glutaraldehyde (1%) to a final glutaraldehyde concentration of 0.02%. The solution was left in a 37° C. oven overnight for gelation and crosslinking.

Dendrimer-crosslinked collagens were prepared using the following procedure. The collagen solution was mixed (˜10 minutes) with an aqueous solution containing EDC, dendrimer and NHS in pre-cooled syringes on ice. The pH of the solution was adjusted to 5.5, the optimal reaction condition for carbodiimide crosslinking [20] and the solution was injected into glass moulds and reacted in a 37° C. oven overnight. The EDC and NHS ratios remained constant based on above. However, different ratios of collagen to dendrimers were studied to examine the effect of dendrimer amount on crosslinking in the resultant gels. The chemical structure of a generation 2 polypropyleneimine octamine dendrimers used for crosslinking is shown in FIG. 5.

The final collagen concentration ranged from 2% to 5% based on different dilution factors. In all cases, due to the high viscosity of the collagen solutions used for gel preparation, it was desirable to avoid the introduction of air into the mixture as this altered the appearance and mechanical properties of the gels formed. Once formed, the resultant gels were removed from the moulds, immersed in glycine solution (0.5% in PBS) at room temperature to neutralize any residual activated carboxylic acid groups and to extract the N-hydroxysuccinimide reaction product, or in the case of the glutaraldehyde-crosslinked gels, to neutralize any residual glutaraldehyde. The final gels were rinsed three times with PBS over a 12 hour period. Prepared gels were stored hydrated in a 4° C. refrigerator until use.

Characterization of Collagen Samples

Transparency Measurements

The collagen samples were examined for transparency by scanning within the visible range of wavelengths (390 nm-780 nm) with Beckman DU-640 spectrophotometer.

Transmission Electron Microscopy (TEM)

Samples were fixed for 2 hours with 2% glutaraldehyde in 0.1M sodium cacodylate buffer (pH7.4), rinsed twice in buffer, post-fixed for 1 hour in a 0.1M sodium cacodylate buffer containing 1% osmium tetroxide, and finally rinsed twice with buffer. Then samples were gradually dehydrated by ethanol (50%, 70%, 95%, 100%) for at least 1 hour at each concentration. The samples were then infiltrated with Spurr's resin through a resin:ethanol series of 1:2, 1:1, 2:1, 100% Spurr's with continuous mixing on a rotator throughout the infiltration process. Once in 100% Spurr's resin, the samples were then cut into blocks of a width of 1 mm, placed into flat embedding moulds and polymerized at 60° C. overnight. The embedded samples were sectioned with a diamond knife on a Leica Ultracut UCT microtome, post-stained with uranyl acetate and then viewed in a JEOL JEM 1200 EX transmission electron microscope operating at 80 kV.

Glucose Permeation

Glucose permeability of the dendrimer-crosslinked collagen gel samples was determined using a custom-made device previously described [29]. Other samples were mechanically not strong enough to be placed into the apparatus without leaking. The glucose concentrations of solutions in the each of the chambers were periodically measured based on the enzymatic conversion of glucose to glucose-6-phosphate followed by production of dinucleotide and quantified UV absorption. The permeability coefficient of glucose in PBS (pH 7.4) was calculated from the rate of glucose concentration change with time.

Mechanical Properties

In order to prepare collagen gel samples for Instron testing, a custom designed mould was prepared. A polymer mesh was incorporated in the gel sample in the area where the gels would be gripped in the test in order to make the handling and gripping of the samples in the testing machine easier as well as to provide an accurate measure of the strength of the gel unaffected by the grips. The area between the grips was free of mesh so that only the gel was tested. Appropriate gel forming solutions were poured into the mould, and the mould was placed under two flat glass plates in order to make the samples. A weight was placed on top of the glass plate to ensure solution contact with the mould and the plates. The mould was then placed overnight in a 37° C. oven under humidified conditions. The gel was removed from the mould and rinsed with Milli-Q water at least three times over 10 hours to remove unreacted crosslinking reagents. The gels were then blotted dry gently with filter paper and mounted on the grips of an Instron Series IX Automated Materials Testing System. A crosshead speed of 5 mm/min and full-scale load range of 500 N were used for the test which was conducted at 23° C. and a humidity of 50%. Young's modulus, maximum load and displacement at maximum load were recorded as indications of the mechanical properties of the various collagen samples.

Suture Strength

Suture strength of collagen gels was also determined since this is anticipated as the location of failure at implantation. A method similar to that recommended for vascular prostheses and triflate heart valves (ANSI/AAMI) was used as suggested previously [30]. Briefly, fully hydrated gels were suspended between two diametrically positioned nylon 10/0 sutures (33 μm diameter), selected based on their used in ocular surgeries, penetrating through the gels at 2 mm from the edge. The free ends of each suture were clamped in the grips of the Instron and the samples were drawn to break at a crosshead speed of 5 mm/min. The suture itself was found to have a breaking load of ˜56 g, which as well above the failure point of the tested gels. The maximum load at breaking was recorded as a very practical indication of gel performance during surgical suturing.

In Vitro Cell Culture Studies

For cell culture, 0.5 cm disks of the gels were exposed to keratinocyte serum-free medium (KSFM, Invitrogen Life Technologies, Burlington ON) containing antibiotics (penicillin/streptomycin 1:100, gentamycin 1:1000). Immortalized human corneal epithelial cells [8] were used to evaluate corneal epithelial compatibility of the various collagen surfaces. The cells were seeded on the gels at a density of 10⁴ cells per well. The cells were incubated for approximately 15 minutes to allow the cells to adhere to the surfaces before keratinocyte serum-free medium containing epidermal growth factor (5 ng/mL) was added. Medium was replaced every two days and the surfaces were examined daily by light microscopy. To quantify cell adhesion and growth, a CYQUANT assay (Molecular Probes, Invitrogen Life Technologies, Burlington ON) was performed at specified times.

Results

Collagen Gel Preparation

While all of the collagen solutions became gels under the specified reaction conditions, a scaffold for a tissue engineered cornea must be transparent and strong enough to withstand suturing. Unlike the other crosslinking methods, which resulted in gels with varying degrees of transparency, the dendrimer crosslinked collagen samples were all transparent and, relative to the other samples, easy to manipulate.

Transparency Measurements

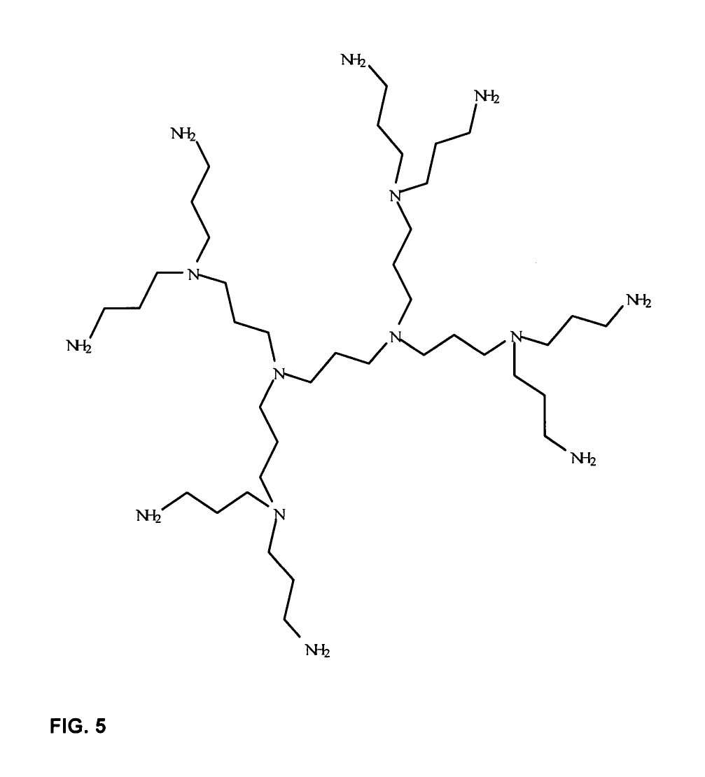

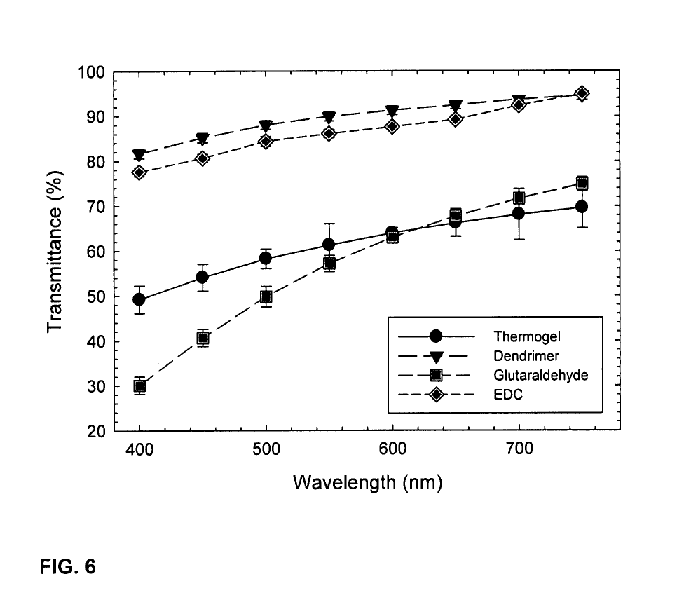

FIG. 6 summarizes light transmission through the samples in the visible light range (390 nm-780 nm), measured as an indication of gel transparency. The EDC and dendrimer-crosslinked collagen samples had very high levels of light transmittance through the entire range of wavelengths. Light transmission through the glutaraldehyde-crosslinked samples was somewhat lower while a significantly (p<0.05) lower level of light transmission was observed with the uncrosslinked thermal gels. This is likely due to fibril formation which is characteristic of this gelling process.

Transmission Electron Microscopy (TEM)

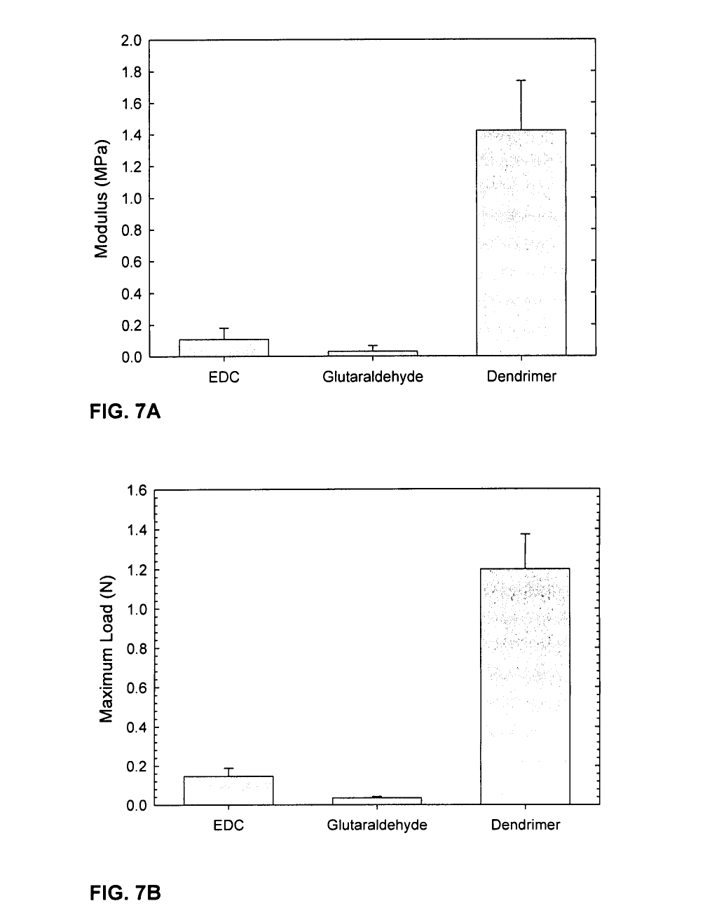

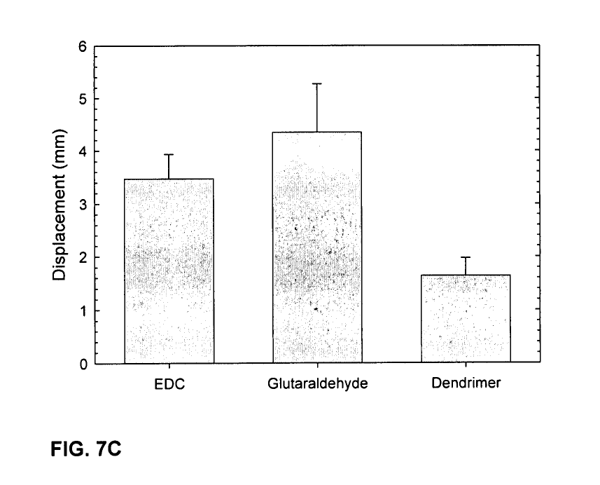

TEM was used to examine gel morphology via the formation of collagen fibers/fibrils during gel preparation as well as the relationship between fibril formation and the crosslinkers. As shown in FIG. 7, no fibrils were observed in the dendrimer-crosslinked collagen samples at a magnification of 20 k. Therefore, these gels will have a high level of transparency. Conversely, in the thermally gelled collagen samples (FIG. 7-a), numerous collagen fibers with a size in the order of 100 nm were noted. Unlike the highly ordered fibril alignment that is present in natural corneal stroma, these fibers were randomly aligned. In the EDC-crosslinked samples, there were few fibrils (FIG. 7-b) although some fine fibrils with sizes on the order of 10-100 nm were observed in the glutaraldehyde-crosslinked samples (FIG. 7-c). Uneven distribution of the fibrils was also observed in these samples, possibly due to the imperfect mixing of the solutions when the samples were prepared.

Glucose Permeability

Since the avascular cornea has a high nutrient permeability, glucose permeability is an important characteristic in corneal tissue engineering scaffolds. The glucose permeability of the corneal stroma has been estimated to be approximately 0.7×10⁻⁶cm²/s [30]. 3% dendrimer-crosslinked collagens had similar or higher glucose permeability at 0.8-1.1×10⁻⁶cm²/s. By decreasing the collagen concentration to 2%, the permeability can be increased to 2.2×10⁻⁶cm²/s.

Mechanical Properties

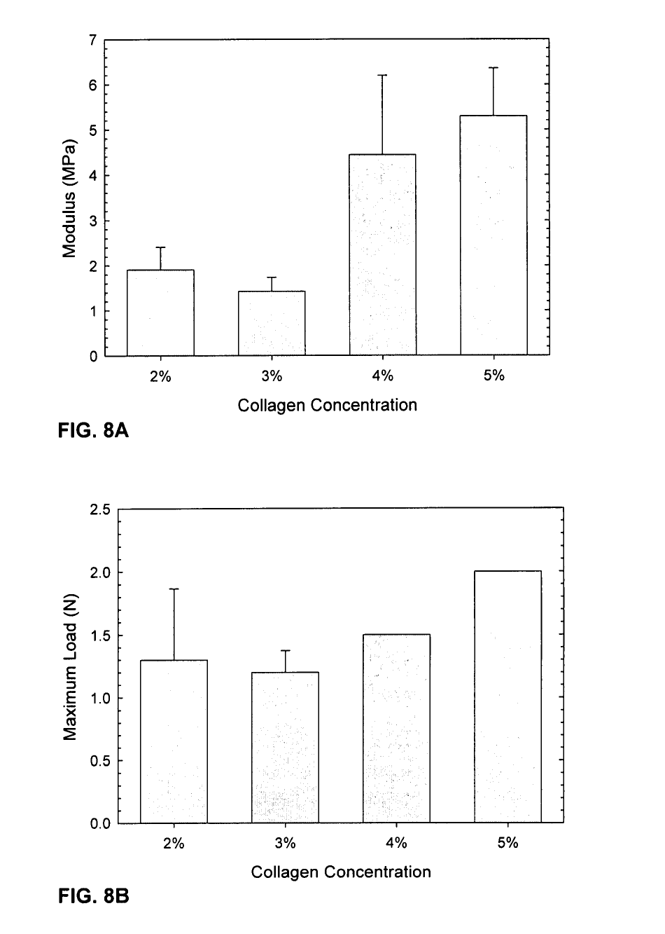

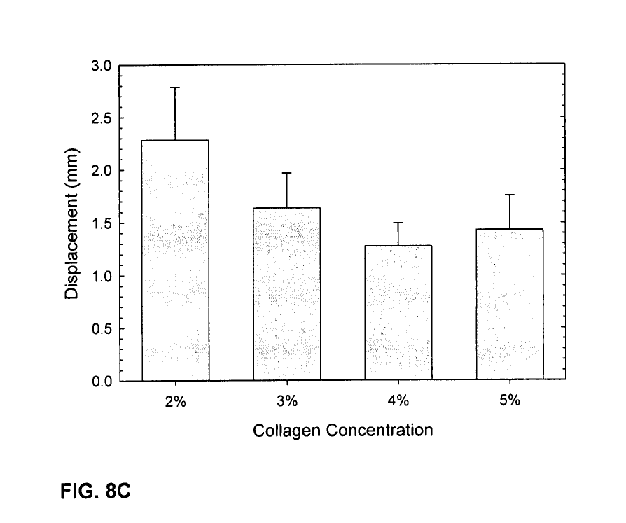

Mechanical properties of collagen gel samples, including Young's modulus, maximum load and displacement at maximum load were measured using an Instron Series IX Automated Materials Testing System. The results for these tests for the various crosslinkers are summarized in FIG. 8. It was not possible to obtain data for the thermally gelled collagen samples as they were extremely weak and did not withstand clamping. Clearly, the dendrimer-crosslinked collagens had the highest Young's modulus at 1.4±0.1 MPa and strength at maximum load at 1.2±0.17 N. These properties in the other samples were at least an order of magnitude lower. As expected, the displacement data showed the opposite trend with the dendrimer-crosslinked collagens having the smallest displacement compared with other samples.

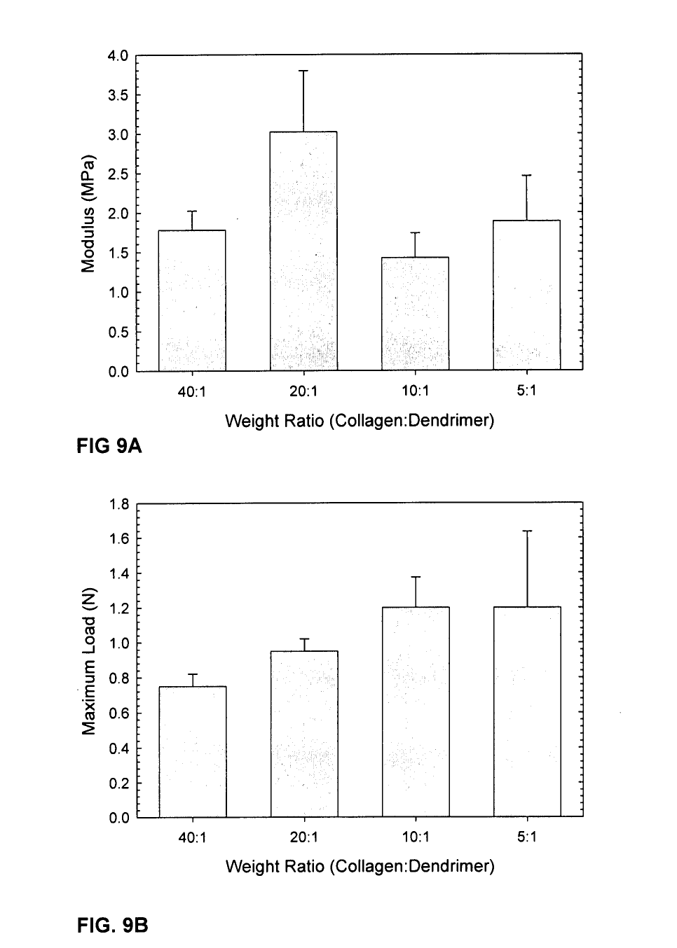

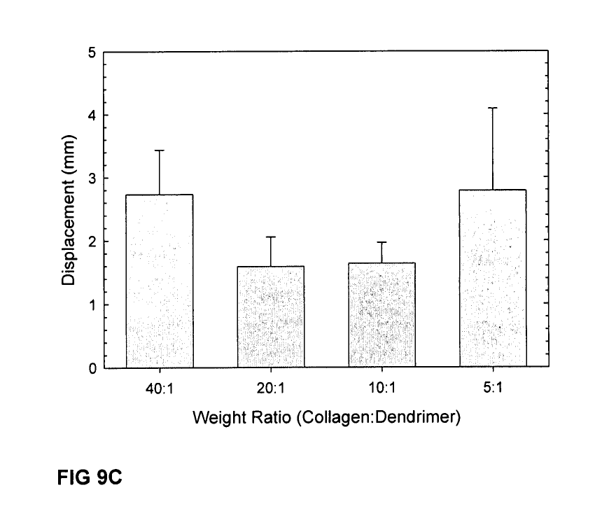

The effect of collagen concentration in the dendrimer-crosslinking reaction of the mechanical properties of the resultant gels was also examined. The results are summarized in FIG. 9. While the trend suggests an increase in Young's modulus and maximum load with increasing collagen concentration and a decrease in displacement (p<0.05), the differences between the 2% and 3% collagen were not significant. While higher concentrations generally resulted in improved mechanical properties, the high viscosity of these samples resulted in mixing difficulties and therefore higher variances in these results. For this reason, all remaining samples were prepared using 3% for the ease of sample handling and consistency.

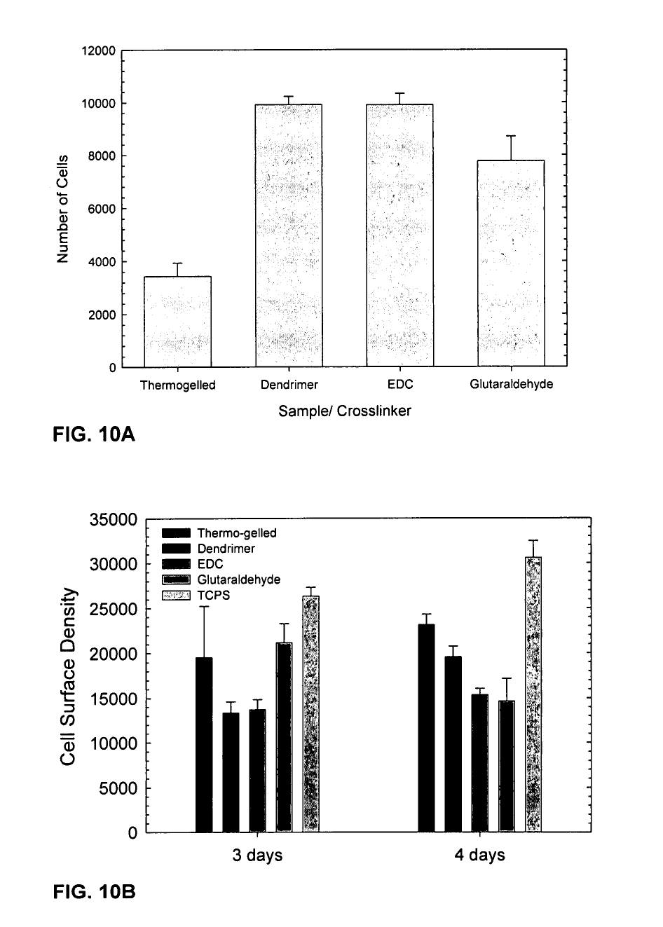

The effect of dendrimer amounts on mechanical properties of dendrimer-crosslinked collagen samples was also examined. As seen in FIG. 10, different collagen to dendrimer weight ratios (40:1, 20:1, 10:1 and 5:1) were used to prepare the samples for the test. Consistent with DSC measurements of denaturation temperature from a previous study [31], increasing the amount of dendrimer in the reaction mixture to amounts greater than stoichiometric had no significant effect on the mechanical properties of the gel (p>0.05).

The effect of another important factor—reaction pH—on the gel properties was also examined and found to not significantly affect Young's modulus although slightly higher values were found with increasing pH (results not shown). However, at pH values above 6.0, the formation of fibrils deteriorated the optical properties of the gels, making them unsuitable for corneal scaffolds. At pH values lower than 5, gelation did not occur.

Suture Strength

Suture strength of the dendrimer-crosslinked collagen gels was also measured as a practical indication of gel performance during surgical suturing. Maximum load of the sutured dendrimer-crosslinked collagen gels was 5.50±0.92 g compared with the strength of nylon 10/0 sutures of ˜56 g. It was also much lower than that of natural cornea, which has a higher strength than that of sutures and which therefore did not break [27,32]. However, it was much higher than the strength of the EDC- and glutaraldehyde-crosslinked samples, which were difficult to suture and could not be placed in the apparatus.

In Vitro Corneal Epithelial Cell Culture

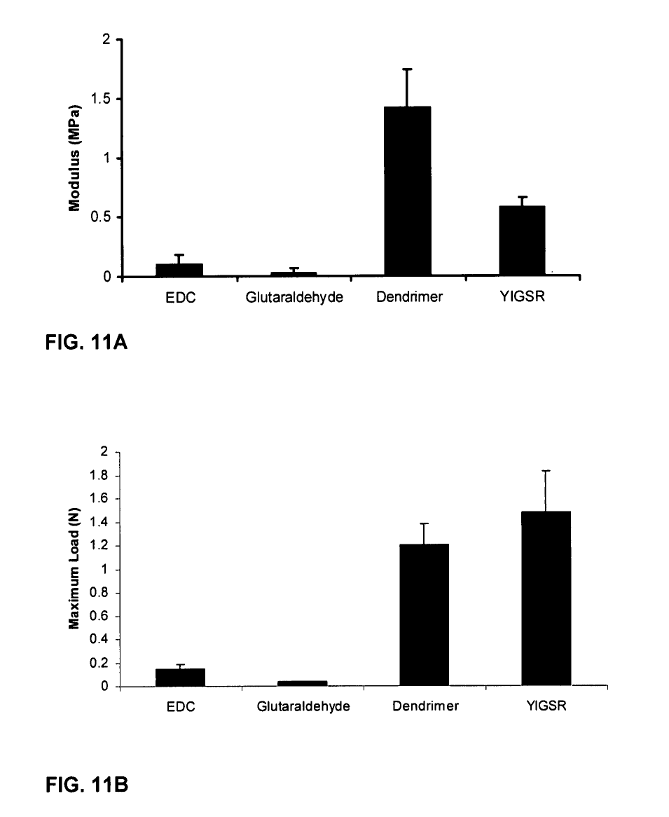

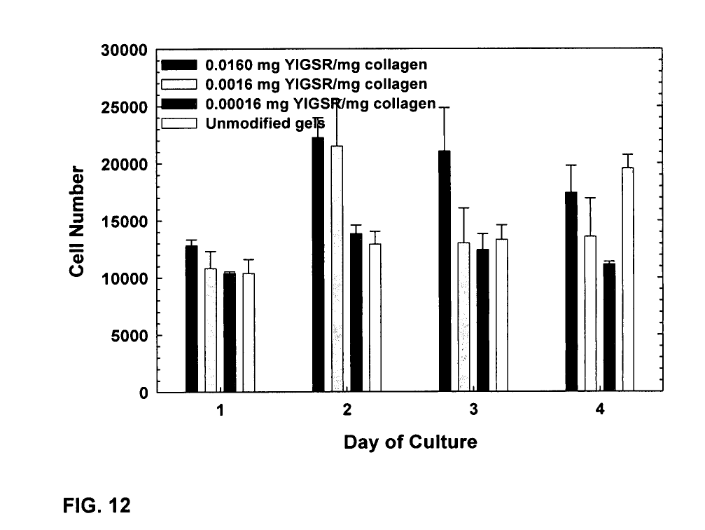

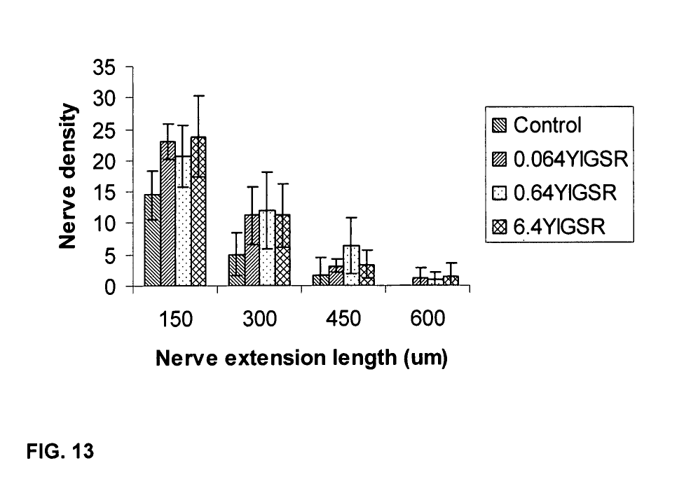

Representative photomicrographs of human corneal epithelial cells on the various surfaces at 120 minutes and on day 4 of culture are shown in FIGS. 11 and 12, respectively. Surprisingly, there are clearly distinct differences in the number of cells present, with the crosslinked gels presumably showing better adhesion than the physically crosslinked thermal gels initially. To better quantify these differences, a Cyquant assay, measuring cell adhesion was performed at short times of 120 minutes (FIG. 13a) and at longer times (3 and 4 days post seeding) (FIG. 13b) to assess cell proliferation. Similar levels of initial adhesion were observed on the EDC- and dendrimer-crosslinked gels and not statistically different (p>0.05). The adhesion of the cells on the glutaraldehyde-modified surfaces was only slightly lower and also not statistically different from the dendrimer and EDC-crosslinked gels. However, that observed on the uncrosslinked thermal gels was much lower (p<0.05). Generally all of the surfaces supported the proliferation of corneal epithelial cells, with similar levels of adhesion at 3 and 4 days post plating. However, it is of interest to note that the glutaraldehyde-crosslinked gels consistently showed decreased cell numbers at 4 days relative to three days, potentially indicative of the release of cytotoxic glutaraldehyde byproducts.

Conclusions

Polypropyleneimine octaamine dendrimers were studied as a means of generating highly crosslinked collagen by amplifying the reaction between collagen molecules using the water-soluble carbodiimide, 1-ethyl-3-(3-dimethyl aminopropyl) carbodiimide hydrochloride (EDC). Compared with EDC only and glutaraldehyde-crosslinked collagens, dendrimer-crosslinked collagen gels had the best optical and mechanical properties. The Young's modulus of the gels was a factor of more than 10 greater with dendrimer-crosslinking compared to EDC-crosslinking. In vitro cell adhesion and growth studies with human corneal epithelial cells show that dendrimer-crosslinking does not adversely affect the biological compatibility of the collagen and suggest that dendrimer-crosslinking may actually result in improved biological interactions. Thus, dendrimers have been successfully applied to collagen crosslinking to produce transparent, mechanically stronger and more biocompatible collagen gels having utility as tissue engineering scaffolds.

### EXAMPLE 3

### Preparation of Collagen Matrix Including Biological Ligands

Covalent Attachment of YIGSR to Dendrimers

YIGSR was added to aqueous dendrimer solutions containing EDC and NHS and reacted overnight at room temperature with stirring. The molar ratio of YIGSR to dendrimer was 1:1, meaning that the number of NH₂ groups for covalent attachment of the peptide was in significant excess and residual amine groups could be used for collagen crosslinking. A ratio of 5:5:1 EDC:NHS:COOH of YIGSR was used. The YIGSR-modified dendrimer product was purified by dialysis with Spectro/Por membrane (MWCO 500) in water for 2 days. The purified product was freeze dried for characterization or further reaction.

Characterization of YIGSR-Modified Dendrimers

The purified YIGSR-modified dendrimer (YIGSR-m-dendrimer) was reconstituted into deuterated water for H-NMR analysis. Spectra for the dendrimer, YIGSR and YIGSR-m-dendrimer were recorded and the peaks compared. MALDI-TOF (Matrix-Assist Laser Desorption Ionization time-of-flight) mass spectrometry was also used to characterize the dendrimer, YIGSR and YIGSR-m-dendrimer. The Micromass TofSpec 2E MALDI-TOF mass spectrometer was operated in reflectron mode using alpha-cyano-4-hydroxycinnamic acid as the matrix. In reflectron mode, an electrostatic mirror bounces the ions back and focuses them at a second detector allowing for better resolution and mass accuracy.

Collagen Gel Preparation

All the reagents used were purchased from Sigma Aldrich (Oakville ON) except when otherwise specified. Concentrated collagen suspensions (6%), the generous gift of Inamed Corporation (USA), contained pepsin digested bovine cornium purified type I collagen predominantly with less than 20% type III collagen. The 6% suspension was in phosphate buffered saline, pH 7.0-7.6. All of the suspensions were acidified with 1N HCl and diluted to make clear collagen solutions prior to further treatment.

Dendrimer-crosslinked collagens were prepared by mixing the collagen solution with an aqueous solution containing EDC, generation 2 polypropyleneimine octaamine dendrimer (FIG. 5), and NHS (molar ratio of EDC:NHS:COOH=5:5:1) in pre-cooled syringes on ice. The pH of the solution was adjusted to 5.5, the optimal reaction condition for carbodiimide-crosslinking [20] and the solution was injected into glass moulds in a 37° C. oven overnight for crosslinking and gelation. 3% collagen gels and a collagen to dendrimer weight ratio of 10:1 were used throughout this study based on previous results [23]. In all cases, due to the high viscosity of the collagen solutions used for gel preparation, the introduction of air into the mixture was avoided as this altered the appearance and mechanical properties of the gels formed. This was achieved by carefully removing air bubbles from the collagen suspensions before they became viscous solutions. YIGSR bulk-modified collagen gels were prepared following the same procedure using a combination of dendrimers with chemically attached YIGSR and unmodified dendrimers as crosslinkers. A series of YIGSR-modified collagen gels with different amounts of YIGSR were prepared by using various YIGSR-m-dendrimer percentages (100%, 10%, 1%) in the crosslinking solution.

Once crosslinked, the gels were removed from the moulds and immersed in glycine solution (0.5% in PBS) at room temperature to react with any residual activated carboxylic acid groups and to extract out the N-hydroxysuccinimide reaction product. The final gels were rinsed with PBS at least three times over a period of 12 hours to remove any residual reaction products. The gels were stored in 4° C. refrigerator. Gels for in vitro cell culture studies were prepared under sterile conditions in a class II biosafety cabinet. All the reagents were either autoclaved or sterilized by filtering with 0.2 μm filters.

Quantification of YIGSR in Collagen Gels

In order to directly quantify the YIGSR content in the modified collagen gels, YIGSR was radiolabeled with ¹²⁵I using Iodogen method [33]. Briefly, ¹²⁵I was added to a YIGSR in a precoated Iodogen vial. After stirring at room temperature for 20 minutes, the labeled YIGSR was purified by dialysis against water using Spectro/Por dialysis membranes (MWCO 500). The radioactivity of the dialysate was monitored until no further free iodide was detected. YIGSR solution containing 10% ¹²⁵I labeled was used to attach to dendrimer and then collagen gel preparation. These collagen gels were counted in a gamma counter to determine the amount of YIGSR in the gels.

YIGSR Surface Modification of the Collagen Gels

Dendrimer only crosslinked collagen gels were immersed in an aqueous solution containing EDC, NHS and the YIGSR. Surfaces with varying peptide coverage were prepared by applying different amounts of peptide. The molar ratio of EDC:NHS:YIGSR was 5:5:1. The pH of the reaction solution was maintained at 5.5 and the reaction was carried out at room temperature overnight with slight agitation. The modified surfaces were thoroughly rinsed with Milli-Q water to remove unreacted peptides and excess EDC and NHS. Surface density of YIGSR was determined using ¹²⁵I radiolabeled peptide.

Surface Characterization of Modified Gels

X-ray photoelectron spectroscopy (XPS) analysis was performed with a Leybold MAX 200×PS System (Cologne, Germany), using a non-monochromatised Mg K_{α} X-ray source operating at 15 kV and 20 mA. The spot size used was 2×4 mm. The energy range was calibrated by placing the Au 4f peak at 84 eV or the main C1s peak at 284.5 eV. Survey scans were performed from 0 to 1000 eV, and low resolution and high resolution C1s spectra were obtained at 90° and 20° takeoff angles of the collagen gels before and after YIGSR modification.

Bulk Characterization of Dendrimer Modified Collagen Gels

Mechanical properties of collagen gels were examined to test the effects of YIGSR modification. In order to prepare collagen gel samples for Instron testing, a custom designed mould was prepared. A polymer mesh was incorporated in the gel sample in the area where the gels would be gripped in the test in order to make the handling and gripping of the samples in the testing machine easier as well as to provide an accurate measure of the strength of the gel that was unaffected by the grips. The area between the grips was free of mesh so that only the gel was tested. Gel forming solution was poured into the mould, and the mould was placed under two flat glass plates in order to make the samples. A weight was placed on top of the glass plate to ensure solution contact with the mould and the plates; otherwise the gel preparation procedure was as with samples for other tests. Prior to testing, the gels were blotted dry gently with filter paper and mounted on the grips of an Instron Series IX Automated Materials Testing System. A crosshead speed of 5 mm/min and full-scale load range of 500 N were used for the test which was conducted at 23° C. and a humidity of 50%. Young's modulus, maximum load and displacement at maximum load were recorded as indications of the mechanical properties of the various collagen samples.

In Vitro Corneal Epithelial Cell Culture Characterization

The response of human corneal epithelial cells to the modified surfaces was examined to assess whether there were differences that resulted from the YIGSR modification. For cell culture, 0.5 cm disks of the sterile gels were pretreated with keratinocyte serum-free medium (KSFM, Invitrogen Life Technologies, Burlington ON) containing antibiotics (penicillin/streptomycin 1:100, gentamycin 1:1000). Immortalized human corneal epithelial cells [Griffith et al., 1999], were seeded on the gels at a density of 10⁴ cells per well. The cells, in a small volume of medium (100-200 μl), were incubated on the surfaces for approximately 15 minutes. This permitted the cells to adhere to the surfaces and ensured that the cells were not washed off the surface of the disks. Following this, epidermal growth factor-containing keratinocyte serum-free medium was added to cover the surfaces. Medium was replaced every two days and the surfaces were examined and photographed daily. To quantify cell adhesion and proliferation, a CYQUANT (Molecular Probes, Invitrogen Life Technologies, Burlington ON) assay was performed at specified times.

In Vitro Early Nerve In-Growth

Early nerve in-growth studies were performed using Dorsal Root Ganglia (DRG) from chicken embryo. Collagen gel samples with varying amounts of incorporated YIGSR were sterilized by incubating in 1% chloroform in PBS for 4 days at 4° C. and subsequently washed in PBS followed by PBS containing antibiotics. Low concentration collagen gels were prepared from diluted collagen solutions for initial adhesion of the DRG. The dorsal root ganglia were isolated from chick embryos and separated from fibroblasts as previously described. Selected DRG's were then dipped into low concentration collagen gels on ice and placed on sample surfaces. Cells were cultured in keratinocyte serum free medium (KSFM) medium (Invitrogen Life Technologies, Burlington ON) supplemented with dexamethasone, dibutryl cyclic adenosine monophosphate (dB cAMP; Sigma), dimethylsulfoxide (DMSO; BDH chemicals). Media was changed every other day. DRG's were allowed to extend for 5 days.

After 5 days of culture, samples were fixed with 4% paraformaldehyde (PFA, Sigma Aldrich, Oakville ON) in PBS. Fluorescent immuno-staining for neurofilament-200 was performed by using mouse monoclonal anti-neurofilament-200 (Sigma Aldrich, Oakville ON) as the primary antibody and fluorescently-labelled goat anti-mouse (Amersham Biosciences) as the secondary antibody. Fluorescent microscopy images were taken of the gels at a magnification of 10 times and a montage was created to show the extension. The numbers of nerves extending 150 μm, 300 μm, 450 μm and 600 μm were counted as a measure of neurite extension.

Results

Collagen Gel Preparation

All the dendrimer crosslinked collagen gels before and after YIGSR peptide modification were transparent and strong enough to manipulate. The gels were stable when stored in PBS/water at 4° C. for at least 8 months.

Covalent Attachment of Peptides to Dendrimers and Characterization

YIGSR and negative control YISGR were attached to dendrimers using the same reaction as was used for dendrimer-mediated collagen crosslinking. The carboxylic acid groups in the peptides were activated by EDC and NHS to form reactive NHS esters, which reacted with amine groups in dendrimers to form chemical bonds.

The reaction between the dendrimers and the peptides was confirmed by H-NMR and MALDI TOF. H-NMR spectra of dendrimer, YIGSR and YIGSR-modified dendrimer are shown in FIG. 14. Characteristic peaks from dendrimers (2.29-2.47 ppm) and YIGSR (6.62-6.91 ppm) were found in the purified YIGSR modified dendrimer spectra, which indicated the successful attachment of YIGSR to dendrimers. The estimated molar ratio of YIGSR:dendrimer was found to be 1:5.4. Therefore, compared with the initial molar ratio of YIGSR:dendrimer (1:1), it is estimated that only 18.5% of the initial YIGSR present was attached to the dendrimers after reaction and purification. Assuming all the YIGSR-modified dendrimers are involved in the crosslinking reaction of collagens, the maximum YIGSR content in the collagen gels would be expected to be 1.6×10⁻² mg/mg collagen.

MALDI mass spectra of dendrimer, YIGSR and YIGSR-modified dendrimer further confirmed the formation of YIGSR-modified dendrimer (FIG. 15). Peaks for the dendrimer (773.7) and YIGSR (595.3) as well as for the YIGSR-modified dendrimer (1354.1) were found in the spectra as expected. Additional peaks present (1186, 1086, 1029, 955) are thought to result from deposition of the chemically-attached YIGSR due to its thermally labile nature [34].

Incorporation of Peptides in Collagen Gels and Characterization of Peptide Modified Gels

¹²⁵I labeled YIGSR were used to quantify the amount of YIGSR incorporated within the collagen gels. It was found that 3.1−3.4×10⁻² mg of YIGSR/mg collagen could be incorporated, suggesting that 24 to 26% of the initial YIGSR present was attached to collagen. This result is consistent with the estimate from the H-NMR analysis of the peptide-modified dendrimers.

Gel Characterization

Surfaces of collagen gels before and after YIGSR modification were examined by XPS. Not unexpectedly, there were no significant differences (data not shown), which demonstrates that the reaction with the peptide-modified dendrimers did not adversely affect the surface properties of the gels. Denaturation temperatures of the collagen gels were determined by DSC to determine whether changes in the crosslinking density occurred with YIGSR modification. Multiple denaturation temperature peaks were found in YIGSR-modified collagen samples similar to the previously examined dendrimer-crosslinked collagens [23]. As shown in Table 3, slightly lower denaturation temperatures were found in YIGSR-modified collagen samples, indicating the possible interference of YIGSR with the dendrimer-mediated crosslinking reaction resulting in a lower crosslinking density. Possibly due to this, slightly poorer mechanical properties in terms of modulus were observed in the YIGSR-modified collagens as shown in FIG. 16a. However, they had a similar maximum load to the unmodified dendrimer-crosslinked collagens (FIG. 16b).

TABLE 3

| | Denaturation | | |
|---|---|---|---|
| Collagens | temp. peak1(° C.) | Peak2 (° C.) | Peak3 (° C.) |
| YIGSR-modified | 56 | 59.8 | 78.2 |
| Control | 40 | 82 | 89 |

Surface Modification of Collagen Gels with YIGSR

The surface coverage of YIGSR on the collagen surfaces was in the range of 88.9-95.6 μg/cm². While this accounts for only 5-6% of the maximal YIGSR coverage calculated theoretically from the availability of amine groups, it is much higher than the densities 2.5×10⁻⁵ μg/cm² reported in other studies previously [35].

In Vitro Corneal Epithelial Cell Culture

YIGSR-Bulk Modified Collagens

Representative photographs of human corneal epithelial cells (HCEC) on YIGSR bulk modified/unmodified collagen gel surfaces at 120 minutes are shown in FIG. 17. It was found that the cells adhered to all of the collagen surfaces within 30 to 60 minutes. Furthermore, morphology changes were observed in all cases. In comparison, the cells did not adhere to the control tissue culture plates and remained round and non-adherent after 2 hours of culture. The presence of YIGSR resulted in the formation of clusters and visibly greater levels of cell attachment. Over longer periods of time, there was a trend showing that the cells proliferated faster on collagen gels with higher YIGSR content as shown in FIGS. 18 and 19. This trend was confirmed by Cyquant assay as shown in FIG. 20.

YIGSR-Surface Modified Collagens

Similar to the YIGSR-bulk modified collagens, the cells adhered to all collagen surfaces within 60 minutes and changed morphology. However, as shown in FIG. 19, over a period of 1 week of culture, there was no significant improvement in the adhesion and growth of the cells on these surfaces relative to the unmodified collagen gels.

Dorsal Root Ganglia Neurite Extension

Neurite extension from DRG cells was found to be significantly enhanced by the presence of the YIGSR in the collagen gels. The length of the neurites and number of neurites, summarized in FIG. 21, was significantly (p<0.05) enhanced by the presence of the cell adhesion peptide (see FIG. 22). Surprisingly, there was little or no effect of peptide concentration in the gel, although it is possible that the surface density of the peptide on these surfaces was relatively similar as this was a bulk modification. Visually, it is clear that the nerve density on these materials was also enhanced by the presence of the peptide.

Conclusions

The YIGSR peptide sequence of laminin was either chemically incorporated into the bulk structure of collagen gels or attached onto collagen gel surfaces by way of dendrimers. The structure of YIGSR-modified dendrimer was confirmed by H-NMR, MALDI mass spectra and the amount of YIGSR incorporated in collagen was determined by ¹²⁵I radiolabelling. The incorporation reaction was carried out under mild aqueous conditions at room temperature and the amount of peptide incorporated can be tuned by varying reaction conditions such as the percentage of peptide modified dendrimers in crosslinker solutions for the collagens. The crosslinking density of the collagen gels was slightly affected by the incorporated YIGSR, resulting in small decreases of the modulus of the gels. However, the overall mechanical properties of the gels was not significantly altered. The incorporated YIGSR peptide promoted the growth of the corneal epithelial cells on collagen gel surfaces in both terms of adhesion and proliferation. As well, neurite extension and nerve cell density was enhanced on these materials relative to unmodified or control peptide modified controls, although no effect of peptide concentration was observed.

### REFERENCES

1. Olsen D, Yang C, Bodo M, Chang M, Chang R, Leigh S, Baez J, Carmichael D, Perälä M, Hämäläinen E R, Jarvinen M, Polarek J. Recombinant collagen and gelatin for drug delivery. Adv Drug Delivery Rev 2003; 55:1547-1567.
2. Ruszczak Z. Effect of collagen matrices on dermal wound healing. Adv Drug Delivery Rev 2003; 55:1595-1611.
3. Wallace D G, Rosenblatt J. Collagen gel systems for sustained delivery and tissue engineering. Adv Drug Delivery Rev 2003; 55:1631-1649.
4. Badylak S E. The extracellular matrix as a scaffold for tissue reconstruction Sem Cell Dev Biol 2002; 13:377-383.
5. Lee C H, Singla A, Lee Y. Biomedical applications of collagen Int J Pharm 2001; 221:1-22.
6. Ho H O, Liu C W, Sheu M T. Diffusion characteristics of collagen films. J Controlled Rel 2001; 77:97-105.
7. Pieper J S, van der Kraan P M, Hafmans T, Kamp J, Buma P, van Susante J L C, van den Berg W B, Veerkamp J H, van Kuppevelt T H. Crosslinked type II collagen matrices: preparation, characterization, and potential for cartilage engineering. Biomaterials 2002; 23:3183-92.
8. Griffith M, Hakim M, Shimmura S, Watsky M A, Li F F, Carlsson D, Doillon C J, Nakamura M, Suuronen E, Shinozaki N, Nakata K, Sheardown H. Artificial human corneas: scaffolds for transplantation and host regeneration. Cornea 2002:21:S54-61.
9. Germain L, Auger F A, Grandbois E, Guignard E, Giasson M, Boisjoly H, Guerin S L Reconstructed human cornea produced in vitro by tissue engineering. Pathobiology 1999; 67: 140-47.
10. Li F F, Carlsson D, Lohmann C, Suuronen E, Vascotto S, Kobuch K, Sheardown H, Munger R, Nakamura M, Griffith M Cellular and nerve regeneration within a biosynthetic extracellular matrix for corneal transplantation Proc Natl Acad Sci USA 2003; 100:15346-15351.
11. Boyce S T, Christianson D J, Hansborough J F. Structure of collagen-GAG dermal skin substitute optimized for cultured human epidermal keratinocytes. J Biomed Mater Res 1988; 22:939-57.
12. Jayakrishnan A, Jameela S R. Glutaraldehyde as a fixture in bioprostheses and drug delivery matrixes. Biomaterials 1996; 17: 471-484.
13. Khor E. Methods for the treatment of collagenous tissues for bioprostheses. Biomaterials 1997; 18: 95-105.
14. Lee C R, Grodzinsky A J Spector M. The effects of crosslinking of collagen-glycosaminoglyean scaffolds on compressive stiffness, chondrocyte-mediated contraction, proliferation and biosynthesis. Biomaterials 2001; 22: 3145-3154
15. Moore M A, Chen W M, Philips R E, Bohachevsky I K, McIlroy B K. Shrinkage temperature versus protein extraction as a measure of stabilization of photooxidized tissue. J Biomed Mater Res 1996; 32: 209-214.
16. Moore M A, Bohachevsky I K, Cheung D T, Boyan B D, Chen W M, Bickers R R, McIlroy B K. Stabilization of pericardial tissue by dye-mediated photooxidation. J Biomed Mater Res 1994; 28: 611-618.
17. Boyce S T, Christianson D J, Hansborough J F, Structure of a collagen-GAG dermal skin substitute optimized for cultured human epidermal keratinocytes. J Biomed Mater Res 1988; 22: 939-957.
18. Weadock K S, Miller E J, Keuffel E I Dunn M G, Effect of physical crosslinking methods on collagen-fiber durability in proteolytic solutions. J Biomed Mater Res 1996; 32: 221-226.
19. Rault I, Frei V, Herbage D. Evaluation of different chemical methods for cross-linking collagen gel, films and sponges. J Mater Sci: Mater in Med 1996; 7: 215-221.
20. Olde Damink L H H, Dijkstra P J, van Luyn M J A, van Wachem P B, Nieuwenhuis P, Feijen J. Cross-linking of dermal sheep collagen using a water-soluble carbodiimide. Biomaterials 1996; 17: 765-773.
21. Osborne C S, Reid W H Grant M H. Investigation into the biological stability of collagen/chondroitin-6-sulphate gels and their contraction by fibroblasts and keratinocytes: the effect of crosslinking agents and diamines. Biomaterials 1999; 20: 283-290.
22. Ma L, Gao C, Mao Z, Zhou J, Shen J. Enhanced biological stability of collagen porous scaffolds by using amino acids as novel crosslinking bridges. Biomaterials 2004; 25: 2997-3004.
23. Duan X, Sheardown H. Crosslinking of collagen with dendrimers. J Biomed Mater Res 2005; 75A: 510-518.
24. Itoh, S.; Takakuda, K.; Samejima, H.; Ohta, T.; Shinomiya K. and Ichinose, S. Synthetic collagen fibers coated with a synthetic peptide containing the YIGSR sequence of laminin to promote peripheral nerve regeneration in vivo. J. Mater. Sci.: Mater. in Med. 10, 129 (1999)
25. Surronen, E. J.; Nakamura, M.; Watsky, M. A.; Stys, P. K.; Muller, L.; Munger, R.; Shinozaki, N.; Griffith, M. Innervated human corneal equivalents as in vitro models for nerve-target cell interatetions, FASEB J. 18, 170 (2004)
26. Tong Y. and Shoichet, M. Enhancing the neuronal interaction on fluoropolymer surfaces with mixed peptides or spacer group linkers, Biomaterials 22, 1029 (2001)
27. Li, F.; Carlsson, D.; Lohmann, C.; Suuronen, E.; Vascotto, S.; Kobuch, K.; Sheardown, H.; Munger, R.; Nakamura, M.; Griffith, M. Cellular and nerve regeneration within a biosynthetic extracellular matrix for corneal transplantation, PNAS 100, 15346 (2003)
28. Petite H, Frei V, Huc A, Herbage D. Use of diphenylphosphorylazide for cross-linking collagen-based biomaterials. J Biomed Mater Res 1994; 28:159-165.
29. Liu L, Sheardown H. Glucose permeable poly (dimethyl siloxane) poly (N-isopropyl acrylamide) interpenetrating networks as ophthalmic biomaterials. Biomaterials 2005; 26: 233-244.
30. McCarey B E, Schimidt F H, Modeling glucose distribution in the cornea. Curr Eye Res 1990; 9: 1025-1039.
31. Pollack A, Blumenfield H, Wax M, Baughn R L, Whitesides G M. Enzyme immobilization by condensation copolymerization into cross-linked polyacrylamide gels. J Am Chem Soc 1980; 102: 6324-6336.
32. Li F, Griffith M, Li Z, Tanodekaew S, Sheardown H, Hakim M, Carlsson D J. Recruitment of multiple cell lines by collagen-synthetic copolymer matrices in corneal regeneration. Biomaterials 2005; 26: 3093-3104.
33. Behr, T. M.; Gotthardt, M.; Becker, W.; Behe, M. Radioiodination of monoclonal antibodies, proteins and peptides for diagnosis and therapy, Neuk. Med. 41, 71 (2002)
34. Hillenkamp, F.; Karas, M.; Beavis, R. C.; Chait, B. T. Structural characterization of protein tryptic peptides via liquid chromatography/mass spectrometry and collision-induced dissociation of their doubly charged molecular ions. Anal. Chem., 63, 1193 (1991).
35. Shaw

We claim:

1. A collagen matrix cross-linked with a dendrimer.

2. A collagen matrix as defined in claim 1, comprising a dendrimer selected from the group consisting of an alkyl-diamine, an alkyl dicarboxylic acid and an aldehyde-terminated dendrimer.

3. A collagen matrix as defined in claim 2, wherein the dendrimer is an alkyl-diamine.

4. A collagen matrix as defined in claim 2, wherein the dendrimer comprises greater than 4 functional branching groups.

5. A collagen matrix as defined in claim 3, wherein the dendrimer is at least a generation 2 polypropyleneimine octaamine dendrimer.

6. A collagen matrix as defined in claim 1, additionally comprising a biomolecule.

7. A collagen matrix as defined in claim 6, wherein the biomolecule is selected from the group consisting of a protein, a peptide, a polysaccharide, a glycoprotein, a growth factor, a therapeutic agent and a cell adhesion factor.

8. A method of preparing dendrimer-crosslinked collagen comprising the steps of incubating a collagen solution with a dendrimer solution in the presence of an agent capable of facilitating the linkage between the collagen and dendrimer for a period of time suitable to achieve the desired amount of crosslinking.

9. A method as defined in claim 8, wherein the ratio of collagen to dendrimer is about 10:1.

10. A method as defined in claim 8, wherein the agent is a carbodiimide.

11. A method as defined in claim 10, wherein the agent is selected from the group consisting of EDC and DDC.

12. A method as defined in claim 8, wherein the dendrimer solution comprises a mixture of unmodified dendrimer and modified dendrimer having a biomolecule linked thereto.

13. A method as defined in claim 8, comprising the additional step of incubating the dendrimer-crosslinked collagen with unmodified dendrimer and a biomolecule for a period of time suitable to achieve linkage of the biomolecule to the collagen.

14. A tissue engineering scaffold comprising dendrimer cross-linked collagen.

15. A tissue engineering scaffold as defined in claim 14, additionally incorporating a biomolecule.

16. A tissue engineering scaffold as defined in claim 14, wherein the dendrimer is an alkyl-diamine.

17. A tissue engineering scaffold as defined in claim 14, wherein the dendrimer comprises greater than 4 functional branching groups.

18. A tissue engineering scaffold as defined in claim 14, wherein the dendrimer is at least a generation 2 polypropyleneimine octaamine dendrimer.

19. A tissue engineering scaffold as defined in claim 14, wherein the ratio of collagen to dendrimer is about 10:1.

20. A tissue engineering scaffold as defined in claim 15, wherein the biomolecule is a cell adhesion factor.
